(19) United States
(12) Patent Application Publication
(10) Pub. No.: US 2007/0270859 A1
(43) Pub. Date: Nov. 22, 2007

(54) Orthopedic screw with break away drive

(75) Inventors: Wilder Companioni, Cordova, TN (US); David Erickson, Memphis, TN (US); Dorian Smith, Memphis, TN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(73) Assignee: SDGI Holdings, Inc.

(21) Appl. No.: 11/414,804

(22) Filed: Apr. 28, 2006

(51) Int. Cl. A61B 17/58

(52) U.S. Cl. 606/73

(57) ABSTRACT

Embodiments of break off screws are disclosed, configured so that when one head or screw portion is broken off, a complete screw with head remains.

The present disclosure relates to a screw useful in orthopedic medical applications that includes a break off portion. In particular, screws are disclosed that, following the severance of a head portion, a second head portion is exposed for later use.

Screws of various type are used in a number of applications in orthopedic surgery. In spinal osteosynthesis procedures and instrumentations, for example, screws may be used as anchor members in holding implants to bone tissue, as locking members joining multi-part implants, and in other ways. When used as anchors, the surgeon must be aware of how far in the screw is inserted and the forces the screw is applying to the bone. If screws are overtightened into bone, damage to the bone surface or an imperfect compatibility between the thread of the screw and a hole in the bone can occur. Such difficulties, if substantial enough, can lessen the strength of the connection between screw and bone, and may lead to pull-out of the screw or other malfunction of the implant. Similarly, when used as locking members, overtightening can damage a part against which the screw presses, as by splaying a U-shaped connector or pedicle screw. Further, it has been found that overtightening a screw or similar locking member can result not only in poor performance, or damage to tissue or to another part, but also in an undesired breakage or other failure of the screw or locking member itself.

Measurement of torques on screws can be done with additional instruments such as a torque wrench. However, the precision of such instruments is not always satisfactory. Furthermore, addition of further instruments can make a kit of products or an already sophisticated surgical procedure even more complicated.

### SUMMARY

In certain embodiments, an orthopedic screw is disclosed that includes a first screw portion with a first distal end, a first proximal end having a first head portion that is an external drive head portion, and a threaded region between the first distal and proximal ends. A second screw portion can include a second distal end and a second proximal end having a second head portion, where the second head portion is an internal drive head portion. The second distal end and first proximal end are adapted to separate from one another upon application of a prescribed force to the second head portion. The first head portion may include a star-shaped or hexagonal-shaped head portion, and the second head portion may have an outer diameter greater than a first head portion outer diameter. The screw portions may be coaxially aligned and/or be coupled together at a separation region adapted to shear upon the application of the prescribed force. The portions may be integrally formed together, and may include a non-metallic material such as an implantable grade polymer and/or a radiopaque marker disposed in the first distal end, the first head portion, or disposed proximal to the threaded region. The second screw portion may be non-threaded.

In another embodiment, an elongate bone screw for engaging a vertebral body can include a distal region with a bone engaging tip and a threaded portion extending proximally from the bone engaging tip, an intermediate region having an external drive head portion and a radiopaque marker, and a proximal region with an internal drive head portion. The proximal region may be non-threaded, and may be adapted to separate from the intermediate region when a prescribed rotational force is applied to the internal drive head portion. An orthopedic system may include a bone plate having at least one hole disposed therethrough and adapted to receive a bone screw, and a bone screw as disclosed herein.

Methods disclosed herein include attaching a plate to a vertebral body, including positioning the plate adjacent the vertebral body, inserting a bone screw having a distal region, an intermediate region with an external drive head portion, and a proximal region with an internal drive head portion, though a hole in the plate, rotating the bone screw at least partially into the vertebral body using a tool adapted to engage the internal drive head portion, and separating the proximal region of the bone screw from the intermediate region using a rotational force which exceeds the shear strength of an interface between the proximal region and the intermediate region. Such methods can further include engaging the external drive head portion and further rotating the bone screw into the vertebral body and/or imaging a location of the bone screw relative to the vertebral body by imaging at least one radiopaque marker disposed in the bone screw.

### BRIEF DESCRIPTION OF THE DRAWINGS

FIG. 1 is a perspective view of an embodiment of an orthopedic screw.

FIG. 2 is a side elevational view of the embodiment of FIG. 1.

FIG. 3 is a top plan view of the embodiment of FIG. 1.

FIG. 4 is a bottom plan view of the embodiment of FIG. 1.

FIG. 5 is a partial cross-sectional view of the embodiment of FIG. 1 with additional structure in an implanted state.

FIG. 6 is a side elevational and partial cross-sectional view of an embodiment of an orthopedic screw with a driving tool.

FIG. 7 is a side elevational view of an embodiment of a rod of multiple screws similar to the embodiment shown in FIG. 1.

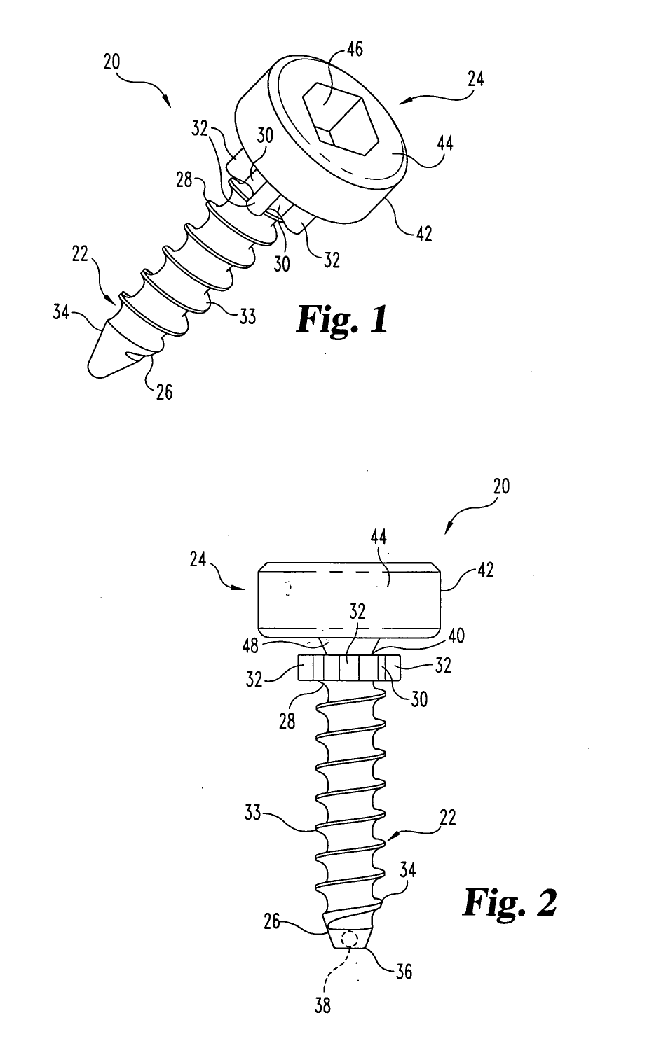

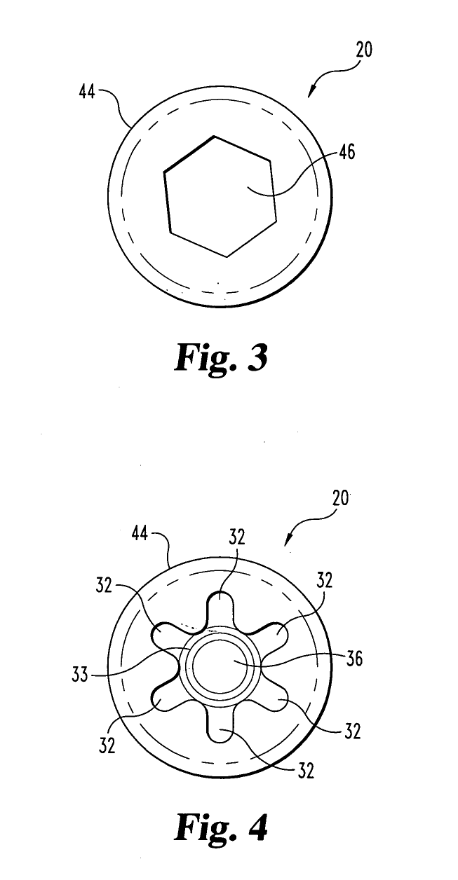

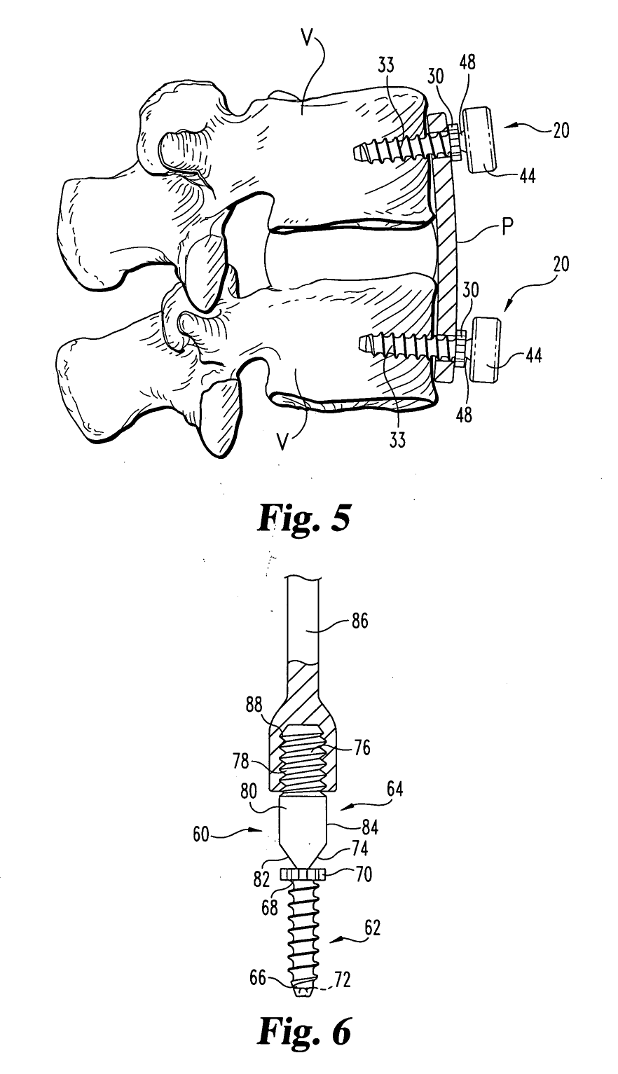

### DESCRIPTION OF THE ILLUSTRATED EMBODIMENTS

For the purposes of promoting an understanding of the principles of the disclosure, reference will now be made to the embodiment illustrated in the drawings and specific language will be used to describe the same. It will nevertheless be understood that no limitation of the scope of the claims is thereby intended, such alterations and further modifications in the illustrated device, and such further applications of the principles of the disclosure as illustrated therein, being contemplated as would normally occur to one skilled in the art to which the disclosure relates.

Referring generally to the figures, there is shown an embodiment of an orthopedic screw 20. Screw 20 may be used in any of a number of orthopedic applications, such as in being inserted into bone and/or other tissue to hold a plate or other implant to such tissue, or for providing a connection between two components of spinal osteosynthesis instrumentation or implants, not shown. For example, screw 20 could be used as a locking member for a multi-axial screw or other implant. Screw 20 may be used in orthopedic surgery in situations where a maximum amount or range of torque or stress on a screw or other component is desired.

In the illustrated embodiment, screw 20 includes a first or distal portion 22 and a second or proximal portion 24, which portions 22 and 24 are at least substantially coaxially aligned. First portion 22 includes a distal end 26 and a proximal end 28 with a head 30 in this embodiment. Head 30 may be configured to be an external drive head, which in a particular embodiment is substantially star-shaped, with six prongs or extensions 32 regularly spaced around proximal end 28 of portion 22. In other embodiments, head 30 may have a different shape (e.g. hexagonal or square), a different number of prongs or extensions or a different spacing of them, or other configurations. Head 30 is intended to accommodate at least a portion of a driving tool (not shown), as further discussed below.

The illustrated embodiment of first portion 22 further includes a threaded portion or shaft 33 between distal end 26 and proximal end 28. Threaded shaft 33 may include cancellous threads for attaching to bone, reverse-angle or standard machine threads for connecting to an implant or implant parts, or other configurations. Shaft 33 has a thread 34 that in the illustrated embodiment extends to or adjoins head 30 of first portion 22. Distal end 26 may have a substantially flat end surface 36, or distal end 26 may be substantially pointed or conic or otherwise configured. For example, distal end 26 may be configured to be self-tapping, particularly in embodiments intended for attachment to bone. At or near distal end 26, a radiopaque member 38 may be placed. In the illustrated embodiment, marker 38 is embedded within shaft 33 adjacent surface 36. In other embodiments, marker 38 could be placed elsewhere in or around shaft 33 and/or its threads, adjacent or on head 30, around surface 36, or in other positions.

Second portion 24, in the illustrated embodiment, includes a distal end 40 and a proximal end 42 with a head portion 44. In this particular embodiment, head portion 44 is an internal drive head that has an outer diameter greater than that of head 30, and that has an internal print 46. Print 46 may be hexagonal, as shown in the drawings, or may be otherwise configured, for example star-shaped, hexalobed (e.g. a TORX print), square, slotted (e.g. single-slot or Phillips-type slots) or threaded, or may have an external hexagonal, threaded or other configuration or print. Second portion 24 includes a shaft portion 48 that extends between head portion 44 and proximal end 28 of first screw portion 22. The illustrated embodiment of shaft portion 48 is not threaded, and is tapered or conical in shape, with the portion adjacent proximal end 28 being of a relative minimum diameter, and the portion adjacent head portion 44 being of a relative maximum diameter. Shaft portion 48 provides a location or region for the separation of portions 22 and 24 from each other, which in the illustrated embodiment abuts head 30 of portion 22. The diameter of the minimum diameter part of shaft 48 can be chosen so that portion 24 will separate from portion 22 on the application of a predetermined or prescribed force or torque. Thus, when a sufficient force or torque is applied to head 44 of portion 24, shaft 48 will break at or adjacent to its minimum diameter, so that portion 24 can be withdrawn and portion 22 will remain where it has been placed.

Examples of uses of screw 20 will now be given in the context of spinal orthopedic surgery. It is to be understood that that context is non-limiting, there being a number of possible orthopedic uses for the disclosed apparatus.

Accordingly, in one embodiment one or more screws 20 can be used to attach a plate member P to one or more vertebrae V. Using a standard, minimally-invasive or other appropriate approach to the vertebrae, a surgeon can position plate P adjacent the vertebrae to be instrumented. Holes may be drilled or otherwise prepared in the vertebrae and/or adjacent tissue, either prior to or after the positioning of plate P. If plate P has pre-existing holes and is positioned first, then plate P may be able to be used as a template or guide for a drill or other instrument for creating holes. The hole(s) may be tapped if necessary or if the surgeon desires. A screw 20 can be inserted through plate P, e.g. through a hole in plate P, and into a corresponding hole in a vertebra V. Screw 20 can be rotated at least partially into vertebra V using a screwdriver (not shown) or other tool adapted to engage screw 20, e.g. via head portion 44. At this point, at least part of shaft 33 of portion 24 is in vertebra V. Portion 24 is then separated from portion 22 through application of force or torque. For example, if shaft 33 is within vertebra V to the extent that head 30 abuts a portion of plate P, then further rotation of portion 24 exerts a rotational force (i.e. a torque) on portion 24 and the connection between portions 22 and 24. When a predetermined torque is reached that exceeds the shear strength of the interface between portions 22 and 24, portions 22 and 24 separate at shaft 48, as discussed above. As another example, rather than plate P providing the resistance to turning that generates the torque, an instrument (not shown) could be used to hold a part of portion 24 (e.g. head 44) while turning force is applied to portion 22 (e.g. head 30).

When portion 24 separates from portion 22, portion 24 can be removed from the surgical site (e.g. by withdrawing the screwdriver or other tool to which portion 22 is connected), leaving portion 22 to anchor plate P to vertebra V. Portion 22 presents an accessible driving or removal head 30 that the surgeon can use to further drive, loosen, or otherwise reposition portion 22 of screw 20 during the current surgery, or in a revision surgery at some future time. For example, the surgeon can engage head portion 30 with an appropriate tool and further rotate portion 22 of screw 20 into vertebra V. The surgeon may also arrange for an x-ray or other imaging method to image the location of screw 20 or portion 22. In embodiments of screw 20 that include radiopaque marker 38, such an image will show marker 38 and thus the position of portion 22, so that the surgeon can verify it to be in the desired location.

It will be seen that other procedures can be performed along with, before or after insertion of screw 20 into a vertebra. For example, plate P can be connected to other implant devices, such as rods, clamps, fusion cages, grafts, spacers or the like. As another example, compression, distraction or rotation, or a combination of those procedures, can be performed on vertebrae V or adjacent vertebrae or other tissue. Once the surgeon has performed all of the procedures he or she desires for the surgery, the surgery can be ended and the wound closed.

As previously noted, other embodiments of screw 20 could be used to connect together parts of other types of implants. As one example, an embodiment of screw 20 could be used as a locking member to hold a spinal rod in a channel in a pedicle anchor (screw, hook or other type) or connector. Once the anchor or connector is in place at the surgical site and the rod is in its channel, shaft 33 of screw 20 can be threaded into the anchor or connector so that end surface 36 abuts the rod. Further application of torque to head 44, as noted above, can generate a torque greater than the shear strength of a portion of screw 20, e.g. shaft 48, which will break and allow portion 24 to be separated and removed from portion 22. Portion 22 will remain in the anchor or connector to lock the anchor or connector to the rod, while presenting an accessible head 30 for further tightening or removal.

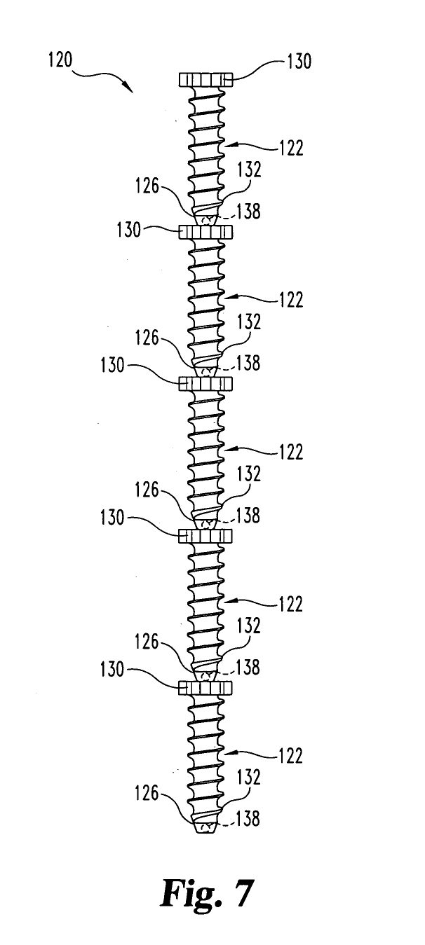

A similar embodiment of a screw 60 is shown in FIG. 6, which includes a first or distal portion 62 and a second or proximal portion 64, which portions 62 and 64 are at least substantially coaxially aligned. First portion 62 is substantially identical, in this embodiment, to the embodiment of portion 22 described above, with a distal end 66, a proximal end 68 with a star-shaped head 70, and a radiopaque marker 72 in this embodiment. It will be seen that portion 62 may be alternatively configured, as indicated herein with respect to portion 22. Second portion 64, like second portion 24 described above, includes a distal end 74 and a proximal end 76 with a head portion 78, which in the illustrated embodiment includes external threads. Second portion 64 includes a shaft portion 80 that extends between head portion 78 and proximal end 68 of portion 62. The illustrated embodiment of shaft portion 80 is not threaded, and has a tapered or conical part 82 and a substantially cylindrical part 84, with the portion adjacent proximal end 68 being of a relative minimum diameter. Shaft portion 80 provides a location or region for the separation of portions 62 and 64 from each other, which in the illustrated embodiment abuts head 70 of portion 62. The diameter of the minimum diameter part of shaft 80 can be chosen so that portion 64 will separate from portion 62 on the application of a predetermined or prescribed force or torque. Thus, when a sufficient force or torque is applied to head 78 of portion 64, as by driving tool 86 with an internally-threaded seat 88, shaft 80 will break at or adjacent to its minimum diameter, so that portion 64 can be withdrawn and portion 62 will remain where it has been placed. Uses of screw 60 can include those described herein with respect to screw 20.

In another embodiment, a rod 120 made of a number of individual screws 122 is provided. Each screw 122 in the embodiment illustrated in FIG. 7 is essentially the same as portion 24, described above. Thus, each screw 122 includes a threaded shaft 132 that adjoins an external driving head 130, that is substantially star-shaped (or TORX-compatible) in a particular embodiment. Head 130 may be thought of as a proximal end in this embodiment, and shaft 132 has a distal end 126. Like portion 24 described above, shaft 132 may have a radiopaque marker 138 at or near distal end 126, or otherwise placed in or around shaft 132.

Screws 122 are connected to each other in head-to-toe fashion, with the head 130 of one screw 122 connected to the distal end 126 of an adjacent screw 122. In this embodiment, screws 122 are integrally formed with one another. In other embodiments, screws 122 may be individually formed and connected together as by weld or other joining method. Distal end 126 may be somewhat conical in configuration, so that a portion adjacent head 130 of another screw 122 has a relatively narrow or minimum diameter, akin to the relative minimum diameter described above with respect to shaft 48 of screw 20.

In use, rod 120 provides a set of screws 122 for rapid insertion in an orthopedic surgical milieu. Rod 120 can be loaded into a quick-load mechanical delivery screwdriver so that a first screw 122, e.g. one to which another screw 122 is not connected at its distal end 126, extends at least slightly from the screwdriver. The screwdriver can be maneuvered to the appropriate location, e.g. adjacent an orthopedic plate member or other implant as discussed above, and the first screw 122 can be inserted into the appropriate aperture. When that screw 122 is tightened to a predetermined torque, it will separate from the next screw 122 at the separation region or narrow portion of distal end 126 of that next screw 122. The screwdriver can be moved from the location of the first screw 122 to be used, and the second screw 122 can move forward in the screwdriver and become available for use. This method can be repeated for each of the screws 122 that make up rod 120. When all screws 122 are used, another rod 120 of screws 122 can be loaded in the screwdriver, if necessary.

It will be seen that screws as described herein can be manufactured from any of a number of biocompatible materials, including stainless steel, titanium or other metals or alloys, certain hard plastics or polymers, ceramics, resorbable materials, or other sturdy materials. Implantable-grade polymers, such as polyetheretherketone (PEEK), are particular examples of non-metallic materials that can be used in manufacturing screws disclosed herein. Screws as disclosed can be made of a single material or of multiple materials. In the illustrated embodiment, portions of screws 20 or 60 or rod 120 are integrally formed with each other, but in other embodiments portions of those items may be made separately and joined together. A joint between them could form a separation region or area where portions (e.g. portions 22 and 24) will separate on application of sufficient force or torque. It will also be seen that head portions of the embodiments disclosed herein may have other shapes and/or forms of attaching to a screwdriver or other tool, and may be of varying size depending on the particular therapy or other use to which they are put.

While the disclosure has been illustrated and described in detail in the drawings and foregoing description, the same is to be considered as illustrative and not restrictive in character, it being understood that only the preferred embodiment has been shown and described and that all changes and modifications that come within the spirit of the disclosure are desired to be protected.

What is claimed is:

1. An orthopedic screw, comprising:
a first screw portion comprising;
a first distal end;
a first proximal end having a first head portion; and
a threaded region between the first distal and proximal ends;
wherein the first head portion is an external drive head portion;
a second screw portion comprising;
a second distal end; and
a second proximal end having a second head portion;
wherein the second head portion is an internal drive head portion; and wherein the second distal end and first proximal end are adapted to separate from one another upon application of a prescribed force to the second head portion.

2. The orthopedic screw as in claim 1 wherein the first head portion comprises a star-shaped screw head portion.

3. The orthopedic screw as in claim 1 wherein the second head portion comprises a hexagonal-shaped screw head portion.

4. The orthopedic screw as in claim 1 wherein the second head portion has an outer diameter greater than a first head portion outer diameter.

5. The orthopedic screw as in claim 1 wherein the first and second screw portions are coaxially aligned.

6. The orthopedic screw as in claim 1 wherein the first and second screw portions are coupled together at a separation region, the separation region adapted to shear upon the application of the prescribed force.

7. The orthopedic screw as in claim 1 wherein the first and second screw portions are integrally formed together.

8. The orthopedic screw as in claim 1 wherein the first and second screw portions comprise a non-metallic material.

9. The orthopedic screw as in claim 8 wherein the non-metallic material is an implantable grade polymer.

10. The orthopedic screw as in claim 8 wherein the first screw portion further comprises at least one radiopaque marker.

11. The orthopedic screw as in claim 10 wherein the at least one radiopaque marker is disposed in the first distal end.

12. The orthopedic screw as in claim 10 wherein the at least one radiopaque marker is disposed in the first head portion.

13. The orthopedic screw as in claim 12 wherein the at least one radiopaque marker is disposed proximal to the threaded region.

14. The orthopedic screw as in claim 1 wherein the second screw portion is a nonthreaded screw portion.

15. An elongate bone screw for engaging a vertebral body, the screw comprising:
a distal region comprising a bone engaging tip and a threaded portion extending proximally from the bone engaging tip;
an intermediate region comprising an external drive head portion and a radiopaque marker; and
a proximal region comprising an internal drive head portion.

16. The elongate bone screw as in claim 15 wherein the proximal region is adapted to separate from the intermediate region when a prescribed rotational force is applied to the internal drive head portion.

17. The elongate bone screw as in claim 15 wherein the proximal region is a non-threaded region.

18. An orthopedic system, comprising:
a bone plate having at least one hole disposed therethrough, the hole adapted to receive a bone screw; and
a bone screw for coupling the bone plate to a bone, the bone screw comprising:
a distal region, an intermediate region, and a proximal region, wherein the intermediate region comprises an external drive head portion and the proximal region comprises an internal drive head portion; and
wherein the proximal region is adapted to separate from the intermediate region when a prescribed force is applied to the internal drive head portion.

19. A method of attaching a plate to a vertebral body, the method comprising:
positioning the plate adjacent the vertebral body;
inserting a bone screw through a hole in the plate, the bone screw comprising a distal region, an intermediate region, and a proximal region, wherein the intermediate region comprises an external drive head portion and the proximal region comprises an internal drive head portion;
rotating the bone screw at least partially into the vertebral body using a tool adapted to engage the internal drive head portion; and
separating the proximal region of the bone screw from the intermediate region using a rotational force which exceeds the shear strength of an interface between the proximal region and the intermediate region.

20. The method as in claim 19 further comprising engaging the external drive head portion and further rotating the bone screw into the vertebral body.

21. The method as in claim 19 further comprising imaging a location of the bone screw relative to the vertebral body by imaging at least one radiopaque marker disposed in the bone screw.

22. An orthopedic screw, comprising:
a first screw portion comprising;
a first distal end;
a first proximal end having a first head portion; and
a threaded region between the first distal and proximal ends;
wherein the first head portion is an external drive head portion;
a second screw portion comprising;
a second distal end; and
a second proximal end having a second head portion;
wherein the second head portion is a threaded drive head portion; and wherein the second distal end and first proximal end are adapted to separate from one another upon application of a prescribed force to the second head portion.
